(19) United States
(12) Patent Application Publication
(10) Pub. No.: US 2019/0014790 A1
(43) Pub. Date: Jan. 17, 2019

(54) DAIRY PRODUCTS FROM HUMAN MOTHER'S MILK

(71) Applicant: Lida Aghdam, COLLEYVILLE, TX (US)

(72) Inventor: Lida Aghdam, COLLEYVILLE, TX (US)

(21) Appl. No.: 15/647,268

(22) Filed: Jul. 12, 2017

(51) Int. Cl. A23C 11/02; A61L 2/10

(52) U.S. Cl. CPC A23C 11/02; A61L 2/10

(57) ABSTRACT

A dairy product, including drinking milk, yogurt, cheese, sour cream, whipping cream, dry milk powder, coffee creamer, ice-cream, kefir, or butter, made from a human mother's milk as its base component. Where the milk has been tested for absence of undesirable chemicals, including prescription medication, illicit drugs, nicotine, and alcohol. With the prescription medication including lithium, chemo therapy agents, Ergotamine, and Bromocriptine, and the illicit drugs including any mind altering substances including marijuana, cocaine, heroin, and Lysergic acid diethylamide (LSD). Where the milk is further exposed to an Ultraviolet (UV) radiation of 260 nm to 270 nm wavelength, disinfecting any undesirable bacteria that was not present in the milk at the time it was expressed. The UV radiation exposure includes placing the milk in a vessel with a mixing mechanism gently agitating the milk, enhancing exposure of a substantial volume of the milk to the UV radiation.

### FIELD OF DISCLOSURE

The present disclosure relates to Dairy Products, and in particular to Dairy Products made from Human Mother's milk.

### BACKGROUND OF THE DISCLOSURE

The milk of animals, and in particular cow milk, goat milk and sheep milk, which are the basis of majority of dairy products in consumption today, are not intended or suitable for human digestion system and in addition to causing gastrointestinal difficulties for a large portion of population it may cause allergic reactions and also unforeseeable medical problems in a minority of dairy consumers.

Because of incompatibility of animals' milk protein with human digestion system, there has been some attempts at reducing side effects of animal milk based dairy products through modifying the animal milk to be lactose-free, milk with added omega-3s, hormone free, and organic and reduced fat milk. In the industrialized countries, animal milk from production to consumption goes through an extensive series of industrial processes to meet government regulations for safety, which will affect the natural state of the milk from the milk that was expressed from the animal in term of pasteurization, heat treatment, and addition of chemicals. Furthermore, dairy product manufacturers using animal milk, depending on the target consumer add sweeteners, preservatives, fruits and fruit essences and nuts to the milk and associated dairy products to make them more appealing to the target consumer.

To satisfy some consumers who do not prefer animal based milk and milk based dairy products, there is a plurality of plant based milk dairy products that are based on soy bean milk, almond milk, coconut milk, hemp milk, rice milk, and some other vegetables or seeds. However for the most part the plant based artificial milk dairy products do not provide the entire ingredient that a baby or adult would get from a milk based diary product.

In general, for people who are allergic to milk, any kind of milk would be off limits. A milk allergy refers to an abnormal immunologic reaction in which the body's immune system produces an allergic antibody, called immunoglobulin E (IgE) antibody, which results in allergy symptoms such as wheezing, diarrhea or vomiting. In particular, symptoms of a milk allergy may include asthma, eczema, gastrointestinal distress, bleeding, pneumonia, and even anaphylaxis (shock).

Some people have sensitivity to the casein (a type of protein) in milk. This sensitivity can trigger inflammation throughout the body, producing symptoms such as sinus congestion, acne flares, skin rash and migraines.

Individuals who are lactose intolerant may be able to tolerate lactose-free milks depending on their lactose intolerance severity. Lactose intolerance is a condition in which a person lacks the enzyme to break down the sugar found in milk for proper digestion. These individuals experience bloating, flatulence or diarrhea when consuming milk and milk products. Lactose-free milk has added enzymes to help with lactose digestion, and may ease or eliminate these symptoms. Levels of lactose intolerance vary per individual. One person as indicated before may tolerate aged dairy with low levels of lactose such as yogurt and hard cheeses. Yet another may be unable to tolerate even a splash of milk in their coffee.

Studies have shown that cow's milk, which is suitable for calves, contains a significant amount of protein. Like all animal protein, cow's milk acidifies the body pH, which in turn triggers a biological correction. Calcium is an excellent acid neutralizer and in the human body the biggest storage of calcium is in the bones. The calcium that body may use to neutralize the acidic body pH may in part affect the calcium storage in the bones. This in turn, in the long term, results in weak bone structure in adults who consume a lot of animal milk. Once calcium is pulled out of the bones, it leaves the body via the urine. The net result may lead to an actual calcium deficit that has detrimental bone health consequences such as Osteoporosis.

Another concern with excess calcium in body and in blood stream is where the calcium may deposit and cause medical anomalies. Such anomalies include the accumulation of calcium in kidney and gallbladder resulting in kidney stone and gallstone, blocking arteries, or Calcium pyrophosphate dihydrate crystal deposition disease (CPPD,) where these crystals form deposits in the joint and surrounding tissues, provoking inflammation in the joint, which can cause the joint cartilage to break down, and lead to a painful inflammatory condition called pseudogout.

Furthermore, nowadays, milking cows are given antibiotics and most are also injected with a genetically engineered form of recombinant bovine growth hormone (rBGH). This man-made or synthetic hormone is used to artificially increase milk production by the cow. The rBGH also increases blood levels of the insulin-growth factor 1 (IGF-1) in those who drink it. Higher levels of IGF-1 are linked to several cancers. One study has shown some direct relation between higher levels of IGF-1 and breast cancer in females. This study has concluded that drinking rBGH milk would thus be expected to significantly increase IGF-1 blood levels and consequently to increase risks of developing breast cancer and promoting its invasiveness.

High levels of IGf-1 also contributes to obesity, chronic diseases like diabetes, hypertension, hyperlipidemia, menstrual irregularities, polycystic ovary disease, insulin resistance, and early onset of menarche.

In some dairy farms dairy cows are kept on sex hormones or pregnant for their entire lives in order to lactate for humans year-round. Consuming milk produced in these dairy farms results in taking in a significant amount of the sex hormones estrogen and progesterone. It is known that an increased exposure to estrogen increases the risk of cancer, especially breast and uterine cancers. Dairy products account for 60 percent to 80 percent of estrogens consumed by humans today.

Individuals who are vegans and avoid animal products would not consume true milk and dairy products made by milk at all and would likely consume plant based milk alternatives and associated dairy products.

It would be desirable to develop dairy products that are not based on animal milk and are an alternative to plant based milk substitutes for adults, children and babies consumption.

### SUMMARY OF THE DISCLOSURE

In order to provide an alternative human tolerable variety of dairy products for adults, children and babies consumption, that provides the required proteins, vitamins and nutrition, and is neither based on animal's milk including cow milk, goat milk and sheep milk, nor it is based on plant based artificial milk including soy bean milk, almond milk, coconut milk, hemp milk, or rice milk; this disclosure details dairy products that are derived from and are based on Human Mother's milk.

In one embodiment a method is disclosed for making a dairy product based on a human mother's milk, where the milk is tested for absence of undesirable chemicals. The undesirable chemicals include prescription medication, illicit drugs, nicotine, and alcohol.

The prescription medication that the milk is tested for, more specifically includes lithium, chemo therapy agents, Ergotamine, and Bromocriptine, which may have been prescribed for and used by the mother who provides the milk. Furthermore, the illicit drugs that are tested for in the milk includes any mind altering substances including marijuana, cocaine, heroin, and Lysergic acid diethylamide (LSD,) which may be used by the mother providing the milk.

The disclosed method of making the dairy products is more specifically related to the dairy products including drinking milk, yogurt, cheese, sour cream, whipping cream, dry milk powder, coffee creamer, ice-cream, kefir, and butter.

The disclosed method further includes a step for disinfecting the milk through exposing the milk to Ultraviolet (UV) light. The exposure of the milk to the UV light is accomplished through containing the milk in a vessel where a mixing mechanism gently agitates the milk while the milk is exposed to an Ultraviolet (UV) light for a predetermined period of time to disinfect the milk and eliminate a plurality of undesirable bacteria present in the milk, where the undesirable bacteria were not originally present in the expressed mother's milk. The process of gently agitating the milk provides for enhanced exposure of a substantial volume of the milk to the UV light, and the UV light has a wavelength of about 260 nm to 270 nm.

In another embodiment a dairy product is disclosed where the dairy products are based on a human mother's milk as the base component, where the milk has been tested for absence of undesirable chemicals. The undesirable chemicals include prescription medication, illicit drugs, nicotine, and alcohol.

The prescription medication that the milk is tested for, more specifically includes lithium, chemo therapy agents, Ergotamine, and Bromocriptine, which may have been prescribed for and used by the mother who provides the milk. Furthermore, the illicit drugs that are tested for in the milk include any mind altering substances including marijuana, cocaine, heroin, and Lysergic acid diethylamide (LSD,) which may be used by the mother providing the milk.

The disclosed dairy products include drinking milk, yogurt, cheese, sour cream, whipping cream, dry milk powder, coffee creamer, ice-cream, kefir, and butter.

The milk that is used for making the dairy product goes through a step for disinfecting the milk through exposing the milk to Ultraviolet (UV) light. The exposure of the milk to the UV light is accomplished through containing the milk in a vessel where a mixing mechanism gently agitates the milk while the milk is exposed to an Ultraviolet (UV) light for a predetermined period of time to disinfect the milk and eliminate a plurality of undesirable bacteria present in the milk, where the undesirable bacteria were not originally present in the expressed mother's milk. The process of gently agitating the milk provides for enhanced exposure of a substantial volume of the milk to the UV light, and the UV light has a wavelength of about 260 nm to 270 nm.

In yet another embodiment a business method is disclosed where a dairy product including a human's mother milk as the base component is offered for sale, where the milk has been tested for absence of undesirable chemicals.

The business method further details that the dairy products offered for sale include drinking milk, yogurt, cheese, sour cream, whipping cream, dry milk powder, coffee creamer, ice-cream, kefir, and butter.

The business method further details that the undesirable chemicals that the milk is tested for, include prescription medication, illicit drugs, nicotine, and alcohol. Here the prescription medication include lithium, chemo therapy agents, Ergotamine, and Bromocriptine, and the illicit drugs include mind altering substances including marijuana, cocaine, heroin, and Lysergic acid diethylamide (LSD).

The business method further details that the milk, that is used for making the dairy products that are offered for sale, goes through a step for disinfecting the milk through exposing the milk to Ultraviolet (UV) light. The exposure of the milk to the UV light is accomplished through containing the milk in a vessel where a mixing mechanism gently agitates the milk while the milk is exposed to an Ultraviolet (UV) light for a predetermined period of time to disinfect the milk and eliminate a plurality of undesirable bacteria present in the milk, where the undesirable bacteria were not originally present in the expressed mother's milk. The process of gently agitating the milk provides for enhanced exposure of a substantial volume of the milk to the UV light, and the UV light has a wavelength of about 260 nm to 270 nm.

### BRIEF DESCRIPTION OF THE DRAWINGS

The disclosure can be better understood with reference to the following detailed description taken in conjunction with the accompanying drawings which illustrate disclosed embodiments. Wherever possible, the same reference numbers will be used throughout the drawing(s) and accompanying written description to refer to the same or like part, wherein:

FIG. 1 is a flow chart describing steps involved in method of making dairy products from human mother's milk, where the milk is collected from a cold storage where the mothers providing the milk were not under medical supervision.

FIG. 2 is a flow chart describing steps involved in method of making dairy products from human mother's milk, where the milk is collected from mothers in a controlled clean room environment, eliminating external contamination of the milk post expression of the milk, where the mothers have not been under medical supervision.

FIG. 3 is a flow chart describing steps involved in method of making dairy products from human mother's milk, where the milk is collected from mothers in a controlled clean room environment, eliminating external contamination of the milk post expression of the milk, and where the mothers have been under medical supervision and are not taking any undesirable medication that can be present in their milk, do not use any tobacco products containing nicotine, do not consume alcohol, and do not use any illicit drugs.

FIG. 4 is a flow chart describing steps involved in a business method for offering for sale a dairy product made from human mother's milk, where the human mother's milk has been tested for absence of undesirable chemicals.

### DETAILED DESCRIPTION

Disclosed embodiments will now be described more fully hereinafter with reference to the accompanying drawings, in which some, but not all of the disclosed embodiments are shown. Several different embodiments are provided and should not be construed as limiting the application of the disclosure to the details of construction and arrangement of the components or method of use set forth in the following description or illustrated in the following drawings as to the embodiments set forth herein. Rather, these embodiments are provided so that this disclosure will be thorough and fully convey the scope of the disclosure to those skilled in the art.

Majority of dairy products consumed presently is derived from the milk of animals, and in particular cow milk, goat milk and sheep milk. Animal milk and dairy products made by the same animal milk are not intended or suitable for human digestion system. The animal milk based dairy products not only causes gastrointestinal difficulties for a large portion of population it may also cause allergic reactions and unforeseeable medical problems in a minority of dairy consumers.

To alleviate the problems associated with animals' milk protein incompatibility with human digestion system, the dairy industry has provided some remedies to reduce side effects of animal milk based dairy products through modifying the animal milk to be lactose-free, introducing animal milk with added omega-3s, providing hormone free animal milk, and further more by introducing organic and reduced fat animal milk. In general, the process of preparing the animal milk for consumer consumption includes an extensive series of industrial processes to meet government regulations for safety, which affects the natural state of the animal milk from the milk that was expressed from the animal in term of pasteurization, heat treatment, and addition of chemicals. Furthermore, depending on the target consumer dairy product manufacturers add sweeteners, preservatives, fruits and fruit essences and nuts to the animal milk and associated dairy products to make them more appealing to the target consumer.

As an existing alternative to animal milk products there is a plurality of plant based milk dairy products that are based on soy bean milk, almond milk, coconut milk, hemp milk, rice milk, and some other vegetables or seeds. However for the most part the plant based artificial milk dairy products do not provide the entire ingredient that a baby or adult would get from animal milk based diary product. Furthermore, there are people who are allergic to plant proteins in soy, coconuts and nuts including almonds, and cannot use any plant based artificial milk dairy products.

This disclosure details using human mother's milk for making an alternative family of dairy products that do not have the harmful effects of animal milk and provide a majority of nutritional and biological ingredients of a milk product that is fully compatible with human digestive system and is specifically made for human consumption including adults, children and babies.

A subsection of the population who has a particular interest in dairy products made from mother's milk are mothers of babies. A subsection of mothers' population, for medical reasons, professional responsibilities, carrier requirements, or simply due to insufficient milk production for nursing, needs alternatives for feeding their babies. Most prefer a natural nutrition separate from animal milk products such as cow milk based formula or soy milk formula, and can benefit from milk and dairy products made from natural human mother's milk.

The family of dairy products made from human mother's milk includes drinking milk, yogurt, cheese, sour cream, whipping cream, dry milk powder, coffee creamer, ice-cream, kefir, and butter.

This disclosure details some specific aspects of making a dairy product based on a human mother's milk. More specifically one aspect of this process involves testing the milk for absence of undesirable chemicals, where the undesirable chemicals include prescription medication, illicit drugs, nicotine, and alcohol.

The testing for prescription medication includes testing for medication that upon consumption by a nursing mother will be present in the mother's milk. These medications include lithium, chemo therapy agents, Ergotamine, and Bromocriptine. Furthermore, the illicit drugs that are tested for in the milk include illicit drugs that if used by the nursing mother will have traces of the drug present in the mother's milk. These illicit drugs include any mind altering substances including marijuana, cocaine, heroin, and Lysergic acid diethylamide (LSD).

Once the milk is tested for absence of undesirable chemicals, the next step is disinfecting the milk through exposing the milk to Ultraviolet (UV) light. The exposure of the milk to the UV light is through placing the milk in a vessel where a mixing mechanism gently agitates the milk while the milk is exposed to an Ultraviolet (UV) light for a predetermined period of time to disinfect the milk and eliminate a plurality of undesirable bacteria present in the milk, where the undesirable bacteria were not originally present in the expressed mother's milk.

Milk is an opaque liquid and as such when exposed to the UV radiation the UV radiation is absorbed through a thin surface layer of the milk accomplishing the bacteria disinfection within the exposed surface of the milk and the underlying layer to the extent that the UV light penetrates the milk. In order to carry out the UV light disinfection of the milk throughout the milk contained in the vessel a mixing mechanism is employed where the milk is agitated and continually the new agitated exposed surface of the milk and the immediate underlying volume includes a volume of milk that has not been exposed to the UV radiation. This process of UV radiation and agitating the milk throughout the UV exposure provides for a substantial volume of the milk to be disinfected through UV light exposure disinfection.

The predetermined period of time for exposing the milk to the UV light depends on the volume of the milk contained in the vessel, intensity of the UV light and distance and angle of exposure of the UV light source with respect to the exposed surface of the milk and vessel configuration. There has been some studies covering UV disinfection of drinking water, however water is a translucent liquid with a different viscosity and chemical characteristic as compared to milk, and as such the mechanism and duration of exposure of the water to the UV light does not correspond to UV light disinfection of an opaque liquid such as milk.

As for characteristics of the UV light used for disinfecting the milk, at certain wavelengths, UV is mutagenic to bacteria, viruses and other microorganisms. In a shortwave wavelength of about 100 to 280 nanometers (nm) the UV radiation is germicidal. In particular at wavelengths around 260 nm to 270 nm, the UV light breaks molecular bonds within microorganismal DNA, producing thymine dimers that can kill or disable the organisms. Depending on the size of microorganism targeted, the exposure time to effectively disable the microorganism varies. It has been shown that the larger microorganisms require longer exposure to be disabled. The exposure time can be a continuous exposure, or an accumulation of repeated intermittent exposures accomplished when as in our case the human mother's milk is circulated under the UV radiation. Accordingly present disclosure provides for utilizing a UV light with a wavelength of about 260 nm to 270 nm for optimum disinfecting of the human mother's milk.

Furthermore, present disclosure details dairy products that are based on a human mother's milk as the base component, where the milk has been tested for absence of undesirable chemicals. The undesirable chemicals include prescription medication, illicit drugs, nicotine, and alcohol.

The disclosed dairy products include drinking milk, yogurt, cheese, sour cream, whipping cream, dry milk powder, coffee creamer, ice-cream, kefir, and butter.

The prescription medication that the milk in the dairy products is tested for includes lithium, chemo therapy agents, Ergotamine, and Bromocriptine, which may have been prescribed for and used by the mother who provides the milk. The illicit drugs that are tested for in the milk in the dairy product include any mind altering substances including marijuana, cocaine, heroin, and Lysergic acid diethylamide (LSD,) which may be used by the mother providing the milk.

The milk that is the basis of the dairy products goes through a disinfecting process involving exposing the milk to Ultraviolet (UV) light before the human mother's milk is processed into the dairy products. The milk is placed in a vessel where a mixing mechanism gently agitates the milk while the milk is exposed to an Ultraviolet (UV) light for a predetermined period of time to disinfect the milk and eliminate a plurality of undesirable bacteria present in the milk, where the undesirable bacteria were not originally present in the expressed mother's milk. The process of gently agitating the milk provides for an enhanced exposure of a substantial volume of the milk to the UV light, and the UV light has a wavelength of about 260 nm to 270 nm.

In addition to this disclosure detailing a process for making dairy products from human Mother's milk and the dairy products that are based on the mother's milk; this disclosure further discloses a business method where the human mother's milk based dairy products are offered for sale, where the milk used for making the dairy products has been tested for absence of undesirable chemicals.

In the disclosed business method further details that the dairy products offered for sale include drinking milk, yogurt, cheese, sour cream, whipping cream, dry milk powder, coffee creamer, ice-cream, kefir, and butter.

The business method further details that the undesirable chemicals tested for in the milk in the dairy products offered for sale include prescription medication, illicit drugs, nicotine, and alcohol. Here the prescription medication include lithium, chemo therapy agents, Ergotamine, and Bromocriptine, and the illicit drugs include mind altering substances including marijuana, cocaine, heroin, and Lysergic acid diethylamide (LSD).

The business method also details that the milk used for making the dairy products that are offered for sale, goes through a step for disinfecting the milk through exposing the milk to Ultraviolet (UV) light. The exposure of the milk to the UV light is accomplished through containing the milk in a vessel where a mixing mechanism gently agitates the milk while the milk is exposed to an Ultraviolet (UV) light for a predetermined period of time to disinfect the milk and eliminate a plurality of undesirable bacteria present in the milk, where the undesirable bacteria were not originally present in the expressed mother's milk. The process of gently agitating the milk provides for enhanced exposure of a substantial volume of the milk to the UV light, and the UV light has a wavelength of about 260 nm to 270 nm.

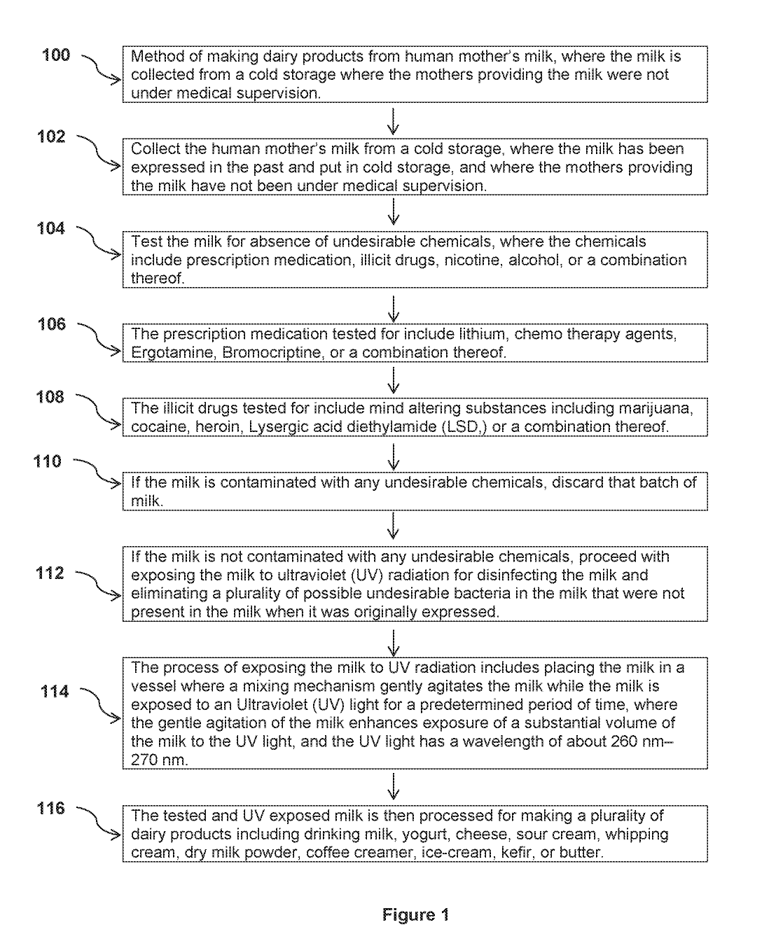

Now in reference to figures provided in this disclosure, FIG. 1 as stated in step 100 details steps involved in a method of making dairy products from human mother's milk, where the milk is collected from a cold storage where the mothers providing the milk were not under medical supervision.

Step 102 indicates that the human mother's milk is collected from a cold storage, where the milk has been expressed in the past and put in cold storage, and where the mothers providing the milk have not been under medical supervision.

Step 104 indicates that the milk is tested for absence of undesirable chemicals, where the chemicals include prescription medication, illicit drugs, nicotine, alcohol, or a combination thereof.

Step 106 indicates that the prescription medication tested for include lithium, chemo therapy agents, Ergotamine, Bromocriptine, or a combination thereof.

Step 108 indicates that the illicit drugs tested for include mind altering substances including marijuana, cocaine, heroin, Lysergic acid diethylamide (LSD,) or a combination thereof.

Step 110 indicates that if the milk is contaminated with any undesirable chemicals, that batch of milk is discarded.

Step 112 indicates that if the milk is not contaminated with any undesirable chemicals, the method proceeds with exposing the milk to ultraviolet (UV) radiation for disinfecting the milk and eliminating a plurality of possible undesirable bacteria in the milk that were not present in the milk when it was originally expressed.

Step 114 indicates that the process of exposing the milk to UV radiation includes placing the milk in a vessel where a mixing mechanism gently agitates the milk while the milk is exposed to an Ultraviolet (UV) light for a predetermined period of time, where the gentle agitation of the milk enhances exposure of a substantial volume of the milk to the UV light, and the UV light has a wavelength of about 260 nm to 270 nm.

Step 116 further indicates that the tested and UV exposed milk is then processed for making a plurality of dairy products including drinking milk, yogurt, cheese, sour cream, whipping cream, dry milk powder, coffee creamer, ice-cream, kefir, or butter.

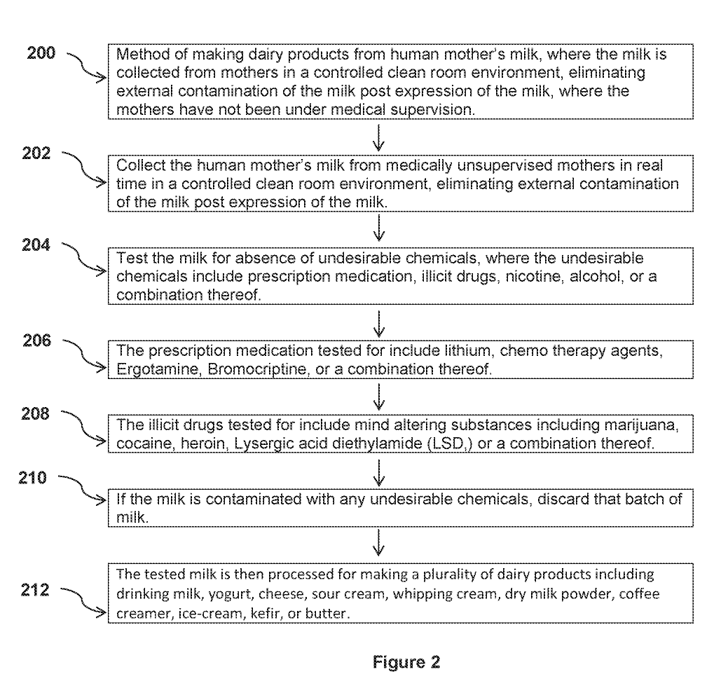

FIG. 2 as stated in step 200 details steps involved in a method of making dairy products from human mother's milk, where the milk is collected from mothers in a controlled cleanroom environment, eliminating external contamination of the milk post expression of the milk, where the mothers have not been under medical supervision.

A cleanroom is a controlled environment that has a low level of pollutants such as dust, airborne microbes, aerosol particles, and chemical vapors. To be exact, a cleanroom has a controlled level of contamination that is specified by the number of particles per cubic meter at a specified particle size. The ambient air outside in a typical city environment contains 35,000,000 particles per cubic meter, some of which are 0.5 mm and larger in diameter.

In general in a cleanroom provisions are made to reduce particulate contamination and control other environmental parameters such as temperature, humidity and pressure as required based on the utilization of the clean room. The key component is a High Efficiency Particulate Air (HEPA) filter that is used to trap particles that are 0.3 micron and larger in size from the air entering the clean room. All of the air delivered to a cleanroom passes through HEPA filters, and in some cases where stringent cleanliness performance is necessary, Ultra Low Particulate Air (ULPA) filters are used. Depending on the class of the clean room, required entry into the clean room may be through pressurized double entry chambers, and personnel may be required to have clean room attire on. In less stringent environments wearing shoe cover booties, going through a high air flow chamber to remove any loose particles from clothing or the person, and an air distribution in the clean room providing for a positive air pressure inside the room may be sufficient for the specific utility of the clean room.

Step 202 indicates that the human mother's milk is collected from medically unsupervised mothers in real time in a controlled cleanroom environment, eliminating external contamination of the milk post expression of the milk.

Step 204 indicates that the milk is then tested for absence of undesirable chemicals, where the undesirable chemicals include prescription medication, illicit drugs, nicotine, alcohol, or a combination thereof.

Step 206 indicates that the prescription medication tested for include lithium, chemo therapy agents, Ergotamine, Bromocriptine, or a combination thereof.

Step 208 indicates that the illicit drugs tested for include mind altering substances including marijuana, cocaine, heroin, Lysergic acid diethylamide (LSD,) or a combination thereof.

Step 210 indicates that if the milk is contaminated with any undesirable chemicals, that batch of milk is discarded.

Step 212 further indicates that the tested milk is then processed for making a plurality of dairy products including drinking milk, yogurt, cheese, sour cream, whipping cream, dry milk powder, coffee creamer, ice-cream, kefir, or butter.

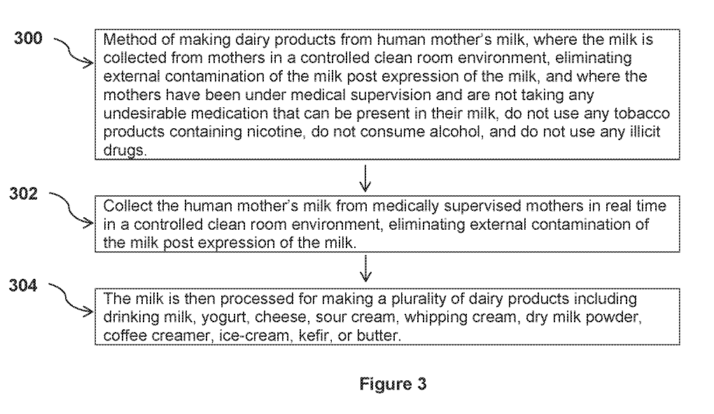

FIG. 3 as stated in step 300 details steps involved in a method of making dairy products from human mother's milk, where the milk is collected from mothers in a controlled cleanroom environment, eliminating external contamination of the milk post expression of the milk, and where the mothers have been under medical supervision and are not taking any undesirable medication that can be present in their milk, do not use any tobacco products containing nicotine, do not consume alcohol, and do not use any illicit drugs.

Step 302 indicates that the human mother's milk is collected from medically supervised mothers in real time in a controlled cleanroom environment, eliminating external contamination of the milk post expression of the milk.

Step 304 further details that the milk is then processed for making a plurality of dairy products including drinking milk, yogurt, cheese, sour cream, whipping cream, dry milk powder, coffee creamer, ice-cream, kefir, or butter.

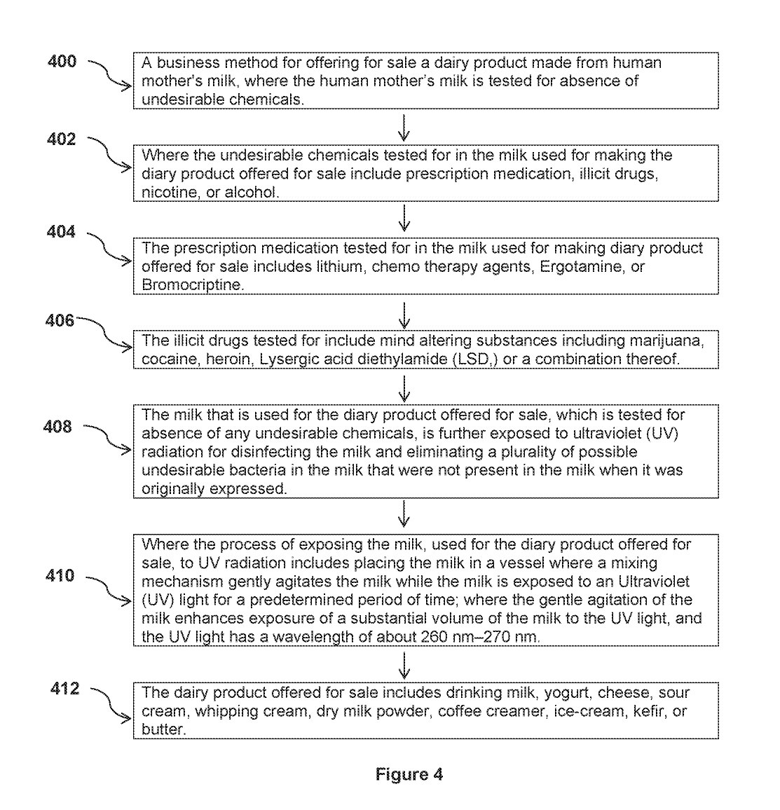

FIG. 4 as stated in step 400 details steps involved in a business method for offering for sale a dairy product made from human mother's milk, where the human mother's milk is tested for absence of undesirable chemicals.

Step 402 details that the undesirable chemicals tested for in the milk used for making the diary product offered for sale include prescription medication, illicit drugs, nicotine, or alcohol.

Step 404 indicates that prescription medication tested for in the milk used for making diary product offered for sale includes lithium, chemo therapy agents, Ergotamine, or Bromocriptine.

Step 406 indicates that the illicit drugs tested for include mind altering substances including marijuana, cocaine, heroin, Lysergic acid diethylamide (LSD,) or a combination thereof.

Step 408 indicates that the milk that is used for the diary product offered for sale, which is tested for absence of any undesirable chemicals, is further exposed to ultraviolet (UV) radiation for disinfecting the milk and eliminating a plurality of possible undesirable bacteria in the milk that were not present in the milk when it was originally expressed.

Step 410 indicates that the process of exposing the milk, used for the diary product offered for sale, to UV radiation includes placing the milk in a vessel where a mixing mechanism gently agitates the milk while the milk is exposed to an Ultraviolet (UV) light for a predetermined period of time; where the gentle agitation of the milk enhances exposure of a substantial volume of the milk to the UV light, and the UV light has a wavelength of about 260 nm to 270 nm.

Step 412 further details that the dairy product offered for sale includes drinking milk, yogurt, cheese, sour cream, whipping cream, dry milk powder, coffee creamer, ice-cream, kefir, or butter.

Many modifications and other embodiments of the disclosure will come to mind to one skilled in the art to which this disclosure pertains having the benefit of the teachings presented in the foregoing descriptions and the associated drawings. The embodiments described herein are meant to be illustrative and are not intended to be limiting or exhaustive. Although specific terms are employed herein, they are used in a generic and descriptive sense only and not for purpose of limitation.

1: A method, comprising:
making dairy products from Human mother's milk,
where the human mother's milk is tested for absence of undesirable chemicals,
where the undesirable chemicals consist of prescription medication consisting of lithium, chemo therapy agents, Ergotamine, and Bromocriptine, illicit drugs, nicotine, and alcohol, and
where the human milk is disinfected through exposure to a UV light with a wavelength of about 260 nm to 270 nm.

2: Method of claim 1, where the undesirable chemicals comprise prescription medication, illicit drugs, nicotine, alcohol, or a combination thereof.

3: Method of claim 1, where the prescription medication comprises lithium, chemo therapy agents, Ergotamine, Bromocriptine, or a combination thereof.

4: Method of claim 1, where the illicit drugs comprise mind altering substances comprising marijuana, cocaine, heroin, Lysergic acid diethylamide (LSD,) or a combination thereof.

5: Method of claim 1, where the dairy products comprise: drinking milk, yogurt, cheese, sour cream, whipping cream, dry milk powder, coffee creamer, ice-cream, kefir, or butter.

6: Method of claim 1, where the human mother's milk is disinfected by being contained in a vessel where a mixing mechanism gently agitates the human mother's milk while the human mother's milk is exposed to the Ultraviolet (UV) light for a predetermined period of time to disinfect the human mother's milk and eliminate a plurality of undesirable bacteria present in the human mother's milk that were not originally present in an expressed human mother's milk.

7: Method of claim 6, where the gentle agitation provides enhanced exposure of the human mother's milk to the UV light.

8: Method of claim 6, where the UV light has a wavelength of about 260 nm to 270 nm.

9: A dairy product, comprising:
a human mother's milk as a base component,
where the human mother's milk has been tested for absence of undesirable chemicals,
where the undesirable chemicals consist of prescription medication consisting of lithium, chemo therapy agents, Ergotamine, and Bromocriptine, illicit drugs, nicotine, and alcohol, and
where the human milk is disinfected through exposure to a UV light with a wavelength of about 260 nm to 270 nm.

10: Dairy product of claim 9, where the undesirable chemicals comprise prescription medication, illicit drugs, nicotine, alcohol, or a combination thereof.

11: Dairy product of claim 9, where the prescription medication comprises lithium, chemo therapy agents, Ergotamine, Bromocriptine, or a combination thereof.

12: Dairy product of claim 9, where the illicit drugs comprise mind altering substances comprising marijuana, cocaine, heroin, Lysergic acid diethylamide (LSD,) or a combination thereof.

13: Dairy product of claim 9, where the dairy products comprise: drinking milk, yogurt, cheese, sour cream, whipping cream, dry milk powder, coffee creamer, ice-cream, kefir, or butter.

14: Dairy product of claim 9, where the human mother's milk is disinfected by being contained in a vessel where a mixing mechanism gently agitates the human mother's milk while the human mother's milk is exposed to the Ultraviolet (UV) light for a predetermined period of time to disinfect the human mother's milk and eliminate undesirable bacteria present in the human mother's milk that were not originally present in an expressed human mother's milk.

15: Dairy product of claim 14, where the gentle agitation provides enhanced exposure of the human mother's milk to the UV light.

16: Dairy product of claim 14, where the UV light has a wavelength of about 260 nm to 270 nm.

17: A business method, comprising:
Offering for sale a dairy product including a human mother's milk as a base component,
where the human mother's milk has been tested for absence of undesirable chemicals,
where the undesirable chemicals consist of prescription medication consisting of lithium, chemo therapy agents, Ergotamine, and Bromocriptine, illicit drugs, nicotine, and alcohol, and
where the human milk is disinfected through exposure to a UV light with a wavelength of about 260 nm to 270 nm.

18: Business method of claim 17, where the dairy product comprise: drinking milk, yogurt, cheese, sour cream, whipping cream, dry milk powder, coffee creamer, ice-cream, kefir, or butter.

19: Business method of claim 17, where the illicit drugs comprise mind altering substances comprising marijuana, cocaine, heroin, Lysergic acid diethylamide (LSD,) or a combination thereof.

20: Business method of claim 17, where the human mother's milk is disinfected by being contained in a vessel where a mixing mechanism gently agitates the human mother's milk while the human mother's milk is exposed to the Ultraviolet (UV) light for a predetermined period of time to disinfect the human mother's milk and eliminate undesirable bacteria present in the human mother's milk that were not originally present in an expressed human mother's milk, and
where the gentle agitation provides enhanced exposure of the human mother's milk to the UV light.
